(19) United States
(12) Patent Application Publication
(10) Pub. No.: US 2020/0169854 A1
(43) Pub. Date: May 28, 2020

(54) METHOD, PROBE AND ARRANGEMENT FOR MONITORING AGRICULTURAL PRODUCTS

(71) Applicant: Quanturi Oy, Espoo (FI)

(72) Inventors: Nadine Pesonen, Siuntio (FI); Roland Guerre, Espoo (FI)

(73) Assignee: Quanturi Oy, Espoo (FI)

(21) Appl. No.: 16/070,566

(22) PCT Filed: Jan. 18, 2017
(86) PCT No.: PCT/FI2017/050020

§ 371 Date: Jul. 17, 2018

(30) Foreign Application Priority Data
Jan. 19, 2016 (FI) 20165032

(51) Int. Cl. H04W 4/38; A01F 25/16; A01F 15/08; G01S 5/04; H04W 4/029; H04Q 9/00; C12M 1/36

(52) U.S. Cl. CPC H04W 4/38; A01F 25/16; A01F 15/08; H04Q 2213/003; H04W 4/029; H04Q 9/00; C12M 41/48; G01S 5/04

(57) ABSTRACT

A method and a probe for monitoring fermentation prone agricultural natural products, such as stored hay, straw, fodder, silage grains, seeds, and kernels. The method includes inserting at least one probe in the product to be monitored. The probe can include at least one sensor for monitoring fermentation, a wireless communication unit for communicating measured data from the probe, sending wirelessly the measured data, receiving the measured data by a base station arranged at a distance from the product, determining the location of the probe by a location unit arranged in the base station, and creating a location data of the probe, and enabling visualization of the measured data and the location data.

### BACKGROUND

The invention relates to a method for monitoring fermentation prone agricultural natural products.

The invention further relates to a probe for monitoring fermentation prone agricultural natural products.

The invention still further relates to an arrangement for monitoring fermentation agricultural natural prone products.

Hay and straw are both agricultural natural products. Hay originates from the cutting and drying of grass, legumes or herbaceous plants. Hay constitutes the main fodder for grazing animals such as cows, horses, sheep, and goats. Straw is the dry stacks of cereal plants and its wide use covers bedding for humans and livestock, biomass, biogas, biofuel, construction materials or crafting. Hay and straw are usually stored as bundles tightly bound with net, wire or twine. Bales may be square, rectangular or round.

In the dairy industry, the price of milk is established based on its microbiological and physio-chemical composition. This composition is directly dependent on the hay quality. A good hay quality directly translates into better milk quality and therefore into higher milk price. Therefore dairy farmers are looking at increasing in priority milk quality rather than milk quantity in order to increase their revenues.

Secondly, it is challenging to know when hay or straw is dry enough for baling or storing as a stack. Farmers manually measure the temperature and humidity of hay e.g. from the cut hay lying on the ground. Some farmers use in-house drying techniques to dry out hay as they are either limited by weather conditions or climate challenges. The end goal is to provide hay with low level of humidity and hence offer digestible and high hay quality. However the drying process is based on a subjective decision process, which rests upon manually measuring the temperature and humidity of only a few hay bales or a few locations of lose hay lying on the ground. There is no information on the complete humidity or temperature distribution of the whole hay stack.

Thirdly, for the equine sector, fodder (hay) quality is of prime importance as it directly affects the performances of horses during e.g. races. It happens that hay is returned to the hay provider by horse owners because of poor hay quality.

Finally, every year, hay stacks and even barns are lost to fires caused by spontaneous auto-combustion, the result of a chemical process that occurs when damp hay heats up and ignites. If hay has not been adequately dried up prior to baling, i.e. if hay bale moisture content is greater than 13-15 percent, the damped compressed hay begins to ferment, a process that builds up heat and produces flammable gases that are at a temperature higher than their ignition point. As the fermentation process continues, hay bale temperature can reach up to 80° C. Passed this threshold any presence of oxygen (e.g. air draft) triggers a spontaneous auto-combustion and hay bale sets on fire resulting in a damaging fire in the storage facility. These fires are not small as they usually involved the whole building. Barn fires result in tremendous financial and psychological and traumas. Globally it is reported that hay fires account for 20 to 35% of barn fires. Preventing methods based on manual temperature and humidity measurements exists but they are proven to be cumbersome, and time consuming. These methods have limited reliability and they only somewhat reduce barn fire risks.

If hay or straw is baled while high moisture content (greater than 13%) or become wet during storage, there are risks of fermentation, which might develop into mould or even into heat and gas, which can ignite causing a phenomenon called auto-combustion.

These two phenomena have direct consequences on farming businesses. Firstly, if moulding occurs, the quality of hay or straw is strongly degraded. Fodder is not as nutritious, has reduced digestibility, increased fibre levels, and less crude protein. Secondly, the consequences of auto-combustion are dramatic as the whole stack and even barn may be lost to fires.

### BRIEF DESCRIPTION

Viewed from a first aspect, there can be provided a method for monitoring fermentation prone agricultural natural products, such as stored hay, straw, fodder, silage grains, seeds, and kernels, the method comprising inserting at least one probe in the product to be monitored, the probe comprising at least one sensor for monitoring fermentation, such as temperature sensor, humidity sensor and/or pH sensor, and a wireless communication unit for communicating measured data from the probe, sending wirelessly the measured data, receiving the measured data by a base station arranged at a distance from the product, determining the location of the probe by a location unit arranged in the base station, and creating a location data of the probe, and enabling visualization of the measured data and the location data.

Thereby a method monitoring the product condition, hence giving information of the status of product, enabling control of product conditions, and therefore improving product quality over time may be achieved.

Viewed from a further aspect, there can be provided a for monitoring fermentation prone agricultural natural products, comprising a casing insertable into the product, at least one sensor for monitoring fermentation, such as temperature sensor, humidity sensor and/or pH sensor, and a communication unit for wireless communication of said measured data from the probe.

Thereby a probe monitoring the product condition, hence giving information of the status of product, enabling control of product conditions, and therefore improving product quality over time may be achieved.

Viewed from a still further aspect, there can be provided an arrangement for monitoring fermentation prone agricultural natural products, the arrangement comprising at least one probe according to any one of claims 5 to 8, a base station comprising a wireless communication unit for receiving measured data from the probe, a location unit for determining the location of the probe, and means for enabling visualization of the measured data and the location data.

Thereby an apparatus monitoring the product condition, hence giving information of the status of product, enabling control of product conditions, and therefore improving product quality over time may be achieved.

Some other embodiments are characterised by what is stated in the other claims. Inventive embodiments are also disclosed in the specification and drawings of this patent application. The inventive content of the patent application may also be defined in other ways than defined in the following claims. The inventive content may also be formed of several separate inventions, especially if the invention is examined in the light of expressed or implicit subtasks or in view of obtained benefits or benefit groups. Some of the definitions contained in the following claims may then be unnecessary in view of the separate inventive ideas. Features of the different embodiments of the invention may, within the scope of the basic inventive idea, be applied to other embodiments.

According to an embodiment, the method comprises sending wirelessly from the base station an output signal based on the measured data and the location data to a centralized server. An advantage is that a remote monitoring can be established and end-users can follow remotely the status of the probe(s) and therefore the status of the material being monitored.

According to an embodiment, the method comprises determining the location of the probe by using triangulation principle using at least three antennas. An advantage is that the probe, and consequently the material being measured can be located according to its position and then methods can be applied to stop or prevent the fermentation process and therefore avoid any potential damage in case of auto-combustion.

According to an embodiment, the method comprises constituting a data network comprising the at least two probes and the base station, and arranging a first probe of said at least two probes for receiving and sending identification codes and measured data of a second probe of said at least two probes. An advantage is that it insures that all the probes are read and it improves the reading distance.

According to an embodiment, the probe comprises an identification unit arranged to store an identification code specific for the probe, and the communication unit being arranged to communicate said identification code from the probe. An advantage is that each probe is uniquely identified.

According to an embodiment, the probe comprises casing comprising an elongated shaft and a tip at the first end of the probe, the elongated shaft comprising an angular outer cross profile, and a handle arranged in the second end of the probe. An advantage is that it minimizes friction and prevents torsion when inserting the probe inside the material.

According to an embodiment, the probe comprises a communication unit comprising a transceiver unit for receiving data wirelessly from another probe, and the communication unit being arranged to communicate said received data from the probe. An advantage is that it insures that all the probes are read and it improves the reading distance.

### BRIEF DESCRIPTION OF FIGURES

Some embodiments illustrating the present disclosure are described in more detail in the attached drawings, in which

FIG. 1 is a schematic view of an arrangement and method,

FIG. 2 is a schematic side view of a probe in partial cross-section,

FIG. 3a is a schematic side view of another probe in partial cross-section, and

FIG. 3b is a schematic cross-sectional view of the probe shown in FIG. 3a.

In the figures, some embodiments are shown simplified for the sake of clarity. Similar parts are marked with the same reference numbers in the figures.

### DETAILED DESCRIPTION

The present disclosure relates to a method, a probe and an arrangement for monitoring fermentation prone products.

The method is using and the arrangement composed of at least one probe and a base station.

The probe contains a sensor selected from temperature sensors, humidity sensors and pH sensors, and a wireless communication unit. The probe(s) is/are inserted in hay storage, inside bale(s) or stack.

The base station communicates with the probe and treats the received data. In an embodiment, the base station stores the data and enables its visualization in real-time. The base station may further transmit wirelessly the measured data as an output signal to a centralized server, such as mobile device, website, or server.

Real-time monitoring enables better product quality control and has a direct impact on preventing barn fires caused by spontaneous combustion.

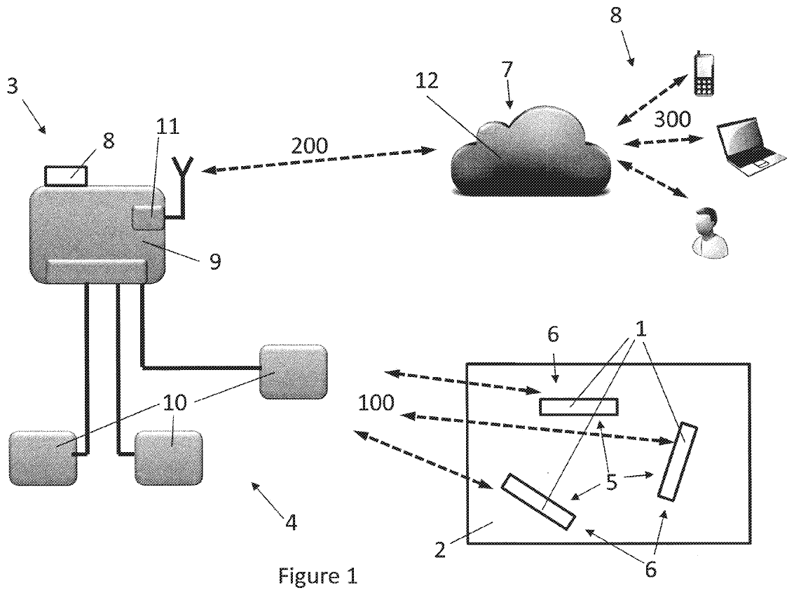

FIG. 1 is a schematic view of an arrangement and method.

According to an idea, the method comprises the following steps:

a) inserting at least one probe 1 in the product 2 to be monitored, the probe 1 comprising at least one sensor 5 for monitoring fermentation, such as temperature sensor, humidity sensor and/or pH sensor, and a wireless communication unit 6 for communicating measured data from the probe 1,

b) sending wirelessly the measured data 100,

c) receiving the measured data 100 by a base station 3 arranged at a distance from the product 2,

d) determining the location of the probe 1 by a location unit 4 arranged in the base station 3 and creating a location data of the probe 1, and

e) enabling visualization of the measured data and the location data.

The embodiment of the method shown in FIG. 1 further comprises three optional steps:

f) sending wirelessly from the base station 3 an output signal 200 based on the measured data and the location data to a centralized server 7,

g) handling the output signal in the centralized server 7 and producing a processed data 300 based on the output signal, and

h) sending wirelessly the processed data 300 to the user interface.

The features of the probe 1 are discussed more detailed later in this description.

The product 2 may be any fermentation prone product, such as stored hay, straw, fodder, silage grains, seeds, and kernels. According to an aspect, the product 2 to be monitored is hay or straw arranged in one or more bales or stored as a stack.

In an embodiment, the base station 3 comprises a central unit 9 that may comprise e.g. a processor (CPU) with a memory configured to store program code and dynamic data. Furthermore, the base station 3 comprises the location unit 4. In the embodiment shown in FIG. 1, the location unit 4 comprises three antennas 10 for data communication with and localization of the probes 1. The localization is based on the triangulation principle that determines the location of an object based on the signal strength response measured from the antennas 4. Localization can further be improved with probes working in a sensor network configuration which is discussed later. Based on the localization, a location data is created in the base station 3, or in the centralized server 7 or in both the base station 3 and the centralized server 7.

According to an idea, the number of the antennas 10 may vary. In an embodiment there are four or even more antennas 10 in the base station 3. The localization may also be realized some other way, too. In an embodiment, it is based on measuring the phase of incoming signals.

In the embodiment shown in FIG. 1, the base station 3 comprises a transmitter unit 11 for sending wirelessly the measured data 100 and a location data for further processing and finally to be shown in a user interface 8. However, in another embodiment there is no transmitter unit 11 at all, but the user interface 8 is arranged in the base station 3.

In an embodiment, the method and arrangement use star topology architecture. This means that each probe 1 sends its data, e.g. temperature and humidity, at set times defined by the base station 3 to the base station 3. In this embodiment the base station 3 is the master and the probes 1 are the slaves. Each of the probes 1 and the base station 3 (if needed, more than one is used) have their own identification number. Anti-collision procedures remove potential transmission errors. In this configuration, the probes 1 have a direct link with the base station 3.

The star topology architecture may also use reflection methods from probe to probe in order to transmit the signal better. A probe 1 may take advantage of the adjacent probe antenna to hop its signal by reflection. Thus the reading distance between the probes 1 and the base station 3 can be increased.

In another embodiment, the method and arrangement use sensor network topology architecture. There is constituted a data network comprising at least two probes 1 and the base station 3, and arranging a first probe 1 of said at least two probes for receiving and sending identification codes and measured data relating to a second probe 1 of said at least two probes. In other words, probes 1 have an ability to operate as transceivers. The probes are capable of receiving and further transmitting the information from an adjacent probe 1. This configuration enables better reliability in reaching out all the probes 1 inserted in the product.

The base station 3 is connecting with the probe(s) 1 from which it detects the strongest signals. These probes 1 further connect to other probes 1 with strong signal until all the probes of the arrangement are connected to a net. This embodiment allows the use of higher frequencies, e.g. ISM band, WiFi, Bluetooth, that have relatively poor penetration inside the product, such as hay. According to an idea, this system may allow auto-reconfiguration of the arrangement in case of additional probes 1 being brought later on.

The output signal 200 is received by the centralized server 7 that is arranged in e.g. cloud or a proprietary hardware. The measured values, e.g. temperature, moisture and/or pH values are gathered in the centralized server where, according to an embodiment, a data analysis is performed in order to create processed data 300 for allowing real time visualization of the monitoring on the user interface 8, e.g. on farmer's computer. The farmer can thus monitor the overall situation of the product 2 in real time. This allows a follow-up of moisture and temperature evolution day by day, and an identification of bales or sections of stack that a prone to fermentation process taking place, etc. With this real-time monitoring, the method and arrangement is able to detect possible combustion to come and to prevent it.

It is to be noted, that the data analysis, or at least part of it, may be performed at the level of the base station 3.

According to an idea, when the temperature rises above a certain threshold value already pre-programmed, an alert may be sent to the user interface 8, with the number of bales at risk and their estimated position within the hay stack/barn, hence allowing their removal from the stack and preventing barn fire.

In an embodiment, and the measured values or the processed data is collected in a database 12. The database 12 enables e.g. retroactive actions such as better knowledge of any potential fermentation process.

Probes 1 are removed just before fodder usage and stored until the following harvesting season. In an embodiment, the probes 1 are recharged automatically during their storage.

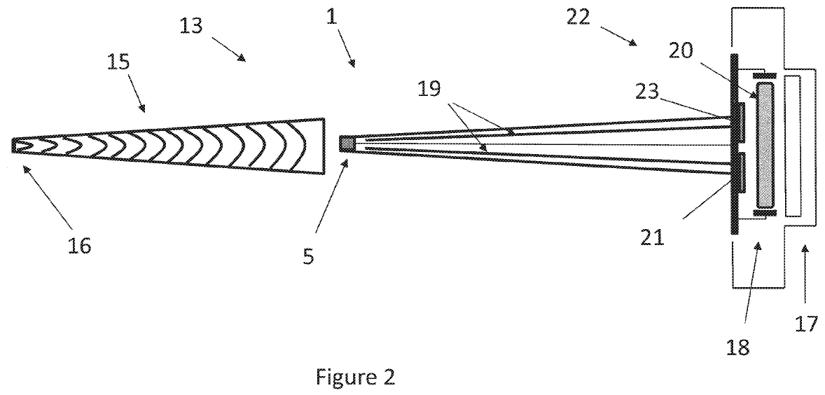

FIG. 2 is a schematic side view of a probe in partial cross-section. The probe 1 can be manually or automatically inserted inside the product to be monitored. The embodiment of the probe 1 shown in FIG. 2 is especially suitable for inserting inside hay or straw or similar bale after the baling process. The design of the probe 1 is optimized for easy penetration inside the non-homogeneous and compact materials as hay and straw. It is to be noted, however, that the shown probe 1 may be used for monitoring another type of products.

The probe 1 comprises a casing 13 insertable into the product, at least one sensor 5 for monitoring fermentation, such as temperature sensor, humidity sensor and/or pH sensor, and a communication unit 6 for wireless communication of said measured data from the probe 1.

The casing 13 of the embodiment shown in FIG. 2 comprises an elongated shaft 15 and a tip 16 at the first end of the probe 1, and a handle 17 arranged in the second end of the probe 1. The casing encloses an electronic circuit 18 therein. In an embodiment, the overall length of the probe 1 is in range of 20-50 cm.

In an embodiment, the casing 13 is made of plastics or plastic composite. The plastics may be e.g. synthetic plastics, such as acrylonitrile butadiene styrene (ABS), polyethylene terephthalate (PET), polyurethane (PU), polycarbonate (PC), polyimide (PI), polyolefin, such as polyethylene (PE) or polypropylene (PP) etc. The material of the casing 13 is preferably food compatible and not prone to oxidation when in contact with damp product. Furthermore, the casing is hermetic and waterproof to at least such an extent that the electronics arranged inside the casing 13 are protected. It is to be noted that the parts of the casing 13 may be manufactured from either similar material or different materials.

The tip 16 allows easy penetration and the wedge-shaped shaft 15 opens up space for the probe as the probe is inserted.

The handle 17 provides easy grasping from the user. In an embodiment, the probe 1 or at least part of the casing 13 is fluorescent or bright colour for easing finding of the probe 1. An identification number or code may be marked on e.g. the handle 17 for recognition of the probe 1.

The electronic circuit 18 is composed of a wireless communication unit 6, a probe antenna 19, a battery 20 and one or more sensors 5. In an embodiment, the wireless communication unit 6 utilizes low frequencies of the ISM band for better radio-frequency penetration inside hay and straw. The frequency may be e.g. 13.56 MHz, 26-28 MHz, 380-390 MHz, 433-435 MHz, 865-930 MHz or 2.4 GHz. The probe antenna 19 is designed for matching the wireless communication unit 6 and for radiating in a possible damped environment.

The electronic circuit 18 reads out the one or more sensors, e.g. temperature, humidity and/or pH sensor(s). It is to be noted, that the probe 1 may also comprise pressure, flow and/or gas etc. sensor(s) which may be used in the method and the arrangement described in this description.

In an embodiment, the electronic circuit 18 has a memory element for data storage and furthermore, the electronic circuit 18 is capable of performing calculations based on the measured parameters.

According to an idea, there is a power saving mode in the electronic circuit 18 to activate/de-activate the sensors during/after a measuring period.

In an embodiment, the electronic circuit 18 has an identification unit arranged to store an identification code specific for the probe 1. The communication unit 6 is arranged to communicate said identification code from the probe 1.

As discussed earlier in this description, the communication unit 6 may comprise a transceiver unit 23 for receiving data wirelessly from another probe, and the communication unit being arranged to communicate said received data from the probe 1 to the base station 3.

In an embodiment, the probe 1 is inserted into the product 2 to be monitored. For instance if the product is hay, the probe 1 is inserted after baling or during unloading of lose hay into barn or similar store. If baling process is used, the probes 1 are manually inserted to the centre part of the bale, or close to it, during the hay bale collection from fields.

This operation is easy and does not require excess strength from the user as the denser part of hay is on the outer side and not in the centre of the bale. This operation is also fast as the user can simply insert the probe 1 one by one once the bales have been collected. This step does not impede the workload or add additional labour costs. Probe-equipped hay bales are then piled up in the barn.

If lose hay is collected from the field, then probes 1 are inserted inside lose hay during unloading. Probes are positioned at different locations in the hay stack.

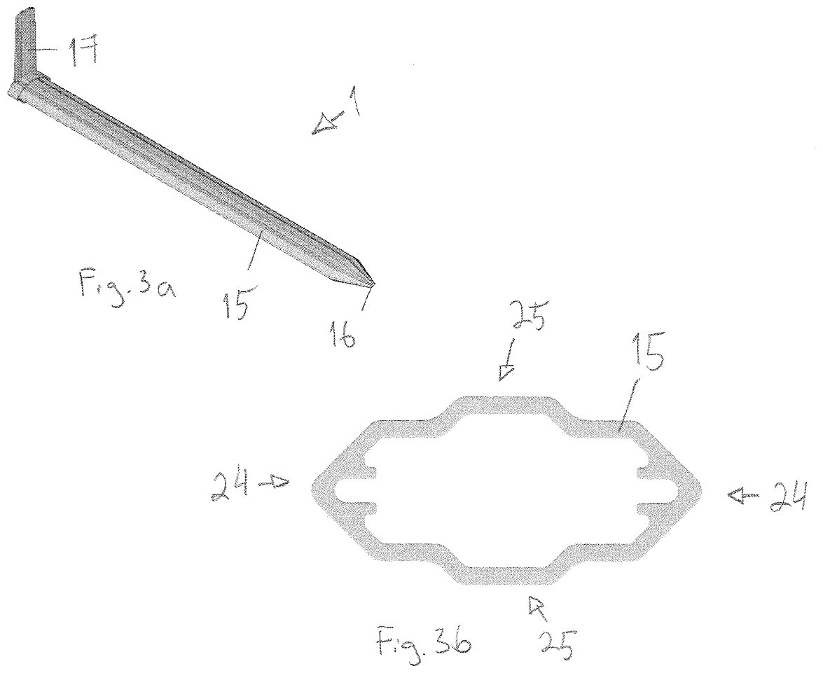

FIG. 3a and FIG. 3b are schematic views of another probe. In principle, this embodiment is similar to that shown in FIG. 2. Now the shaft has an oblong profile with a conical tip 16.

The shaft 15 is flat and comprises an angular outer cross section as shown in FIG. 3b. Angular sides 24 of the shaft 15 push away the fibres when the user pushes the probe forward. This prevents torsion when inserted the probe 1 inside hay or straw bale. In addition, the centre part of the top and bottom sides is reinforced with elevated material sections 25. This prevents torsion when inserting the probe inside the material. The combination of angular sides 24 and reinforced sides 25 also maximizes grip whilst the probe 1 is left inside hay. The probe 1 is meant to stay inside hay during storage and ought to withstand any displacement of the bale. The probe 1 does not drop off when the bale is lifted up with farming equipment such as lifting fork.

The dimensions of the cross profile of the shaft shown in FIG. 3a are in proportion of 1:2 which has been proved to be a good choice for hay and straw. According to an embodiment, said proportion may be selected in range of 1.5:1 to 3:1. It is to be noted, however, that the cross profile may also be round, oval, rectangular, square, polygonal etc.

Overall the design with the oblong profile minimizes the volume occupied when storing several probes.

The embodiments described in this description may have several advantages:

a) Better hay quality management resulting into higher incomes. The price of milk is established based on its microbiological and physio-chemical composition. Hay quality has a direct influence on the quality of produced milk.

b) Enabling recording the product, such as hay or hay bales, history during storage. If the data shows an increase of temperature over time, then it means that the product has suffered from fermentation and moulding will be present. By looking at the history of the product, the farmer can discard the least quality product or use them for other purposes.

c) Drying optimization: temperature and humidity monitoring can help tune and optimize the drying process of products, such as hay, inside in-house dryers. This would give a complete distribution of humidity over e.g. the whole hay stack.

d) Smarter, easier and safer farming by decreasing of life endangering accidents.

e) Product traceability by including in the probe and/or the arrangement memory basic data about the product, such as hay bales. This may include e.g. location and time of baling as well as weather information. Traceability is an important factor for farmers to manage better for instance hay distribution for fodder and constitute an additional guarantee when selling hay.

f) Monitoring of silage quality (moisture) that has a direct impact on milk production and quality.

g) Monitoring temperature and moisture of grain inside silos for e.g. fire prevention.

The invention is not limited solely to the embodiments described above, but instead many variations are possible within the scope of the inventive concept defined by the claims below. Within the scope of the inventive concept the attributes of different embodiments and applications can be used in conjunction with or replace the attributes of another embodiment or application.

The drawings and the related description are only intended to illustrate the idea of the invention. The invention may vary in detail within the scope of the inventive idea defined in the following claims.

### REFERENCE SYMBOLS

1 probe
2 product
3 base station
4 location unit
5 sensor
6 wireless communication unit
7 centralized server
8 user interface
9 central unit
10 antenna
11 transmitter unit
12 database
13 casing
15 shaft
16 tip
17 handle
18 electronic circuit
19 probe antenna
20 battery
21 memory element
22 identification unit
23 transceiver unit
24 angular side
25 elevated material section
100 measured data
200 output signal
300 processed data

1. A method for monitoring fermentation prone agricultural natural products, the method comprising:
inserting at least one probe in a product to be monitored, the probe including a casing insertable into the product, at least one sensor for monitoring fermentation, and a wireless communication unit for communicating measured data from the probe, wherein the casing includes an elongated shaft and a tip at a first end of the probe, the elongated shaft including an angular outer cross profile, and a handle arranged in a second end of the probe;
sending wirelessly measured data;
receiving the measured data by a base station arranged at a distance from the product;
determining a location of the probe by a location unit arranged in the base station, and creating a location data of the probe; and
enabling visualization of the measured data and the location data.

2. The method of claim 1, comprising:
sending wirelessly from the base station an output signal based on the measured data and the location data to a centralized server.

3. The method of claim 1, comprising:
determining the location of the probe by using triangulation principle using at least three antennas.

4. The method of claim 2, comprising:
constituting a data network containing the at least two probes and the base station, and arranging the first probe of said at least two probes for receiving and sending identification codes and measured data relating to a second probe of said at least two probes.

5. The method of claim 1, wherein the fermentation prone product is selected from hay and straw, fodder and silage; grains, seeds, and kernels.

6. A probe for monitoring fermentation prone agricultural natural products, the probe comprising:
a casing insertable into a product;
at least one sensor for monitoring fermentation; and
a communication unit for wireless communication of measured data from the probe;
the casing including an elongated shaft and a tip at a first end of the probe, the elongated shaft including an angular outer cross profile, and a handle arranged in a second end of the probe.

7. The probe of claim 6, comprising:
an identification unit arranged to store an identification code specific for the probe, the communication unit being arranged to communicate said identification code from the probe.

8. (canceled)

9. The probe of claim 6, wherein the communication unit comprises:
a transmitter unit for receiving data wirelessly from another probe, the communication unit being arranged to communicate said received data from the probe.

10. An arrangement for monitoring fermentation prone agricultural natural products, the arrangement comprising:
at least one probe according to claim 6; and
a base station comprising:
a wireless communication unit for receiving measured data from the probe,
a location unit for determining a location of the probe, and
means for enabling visualization of the measured data and the location data.

11. The probe of claim 6, wherein the sensor is one of a temperature sensor, humidity sensor and pH sensor.
